(19) United States
(12) Patent Application Publication
(10) Pub. No.: US 2023/0313117 A1
(43) Pub. Date: Oct. 5, 2023

(54) FLUID FILTRATION SYSTEM

(71) Applicant: Janssen Biotech, Inc., Horsham, PA (US)

(72) Inventors: Hsu-Feng KO, North Wales, PA (US); Douglas KENNEDY, Drexel Hill, PA (US); Richard P. ROHE, Schwenksville, PA (US); Ravinder BHATIA, Blue Bell, PA (US); Bas DIERENBROEK, Utrecht (NL); Lodewijk DE JONGE, Leiden (NL); Phliipp HUBER, Bern (CH); Hans Ulrich DELLENBACH, Thörishaus (CH)

(21) Appl. No.: 18/006,334

(22) PCT Filed: Jul. 19, 2021
(86) PCT No.: PCT/US2021/042168

§ 371 Date: Jan. 20, 2023

Related U.S. Application Data
(60) Provisional application No. 62/705,876, filed on Jul. 20, 2020.

(51) Int. Cl. C12M 1/00; C12M 1/12

(52) U.S. Cl. CPC C12M 47/12; C12M 37/02

(57) ABSTRACT

An improved fluid filtration system is described. The system uses improved sensor assemblies to avoid the problem of biomass build up and reduce the holdup volume of liquid outside of the process vessel. It significantly enhances the performance, robustness and consistency of the filtration.

### CROSS-REFERENCE TO RELATED APPLICATIONS

The present application claims the priority of United States provisional patent application Serial No. 62/705,876 filed Jul. 20, 2020, the contents of which is incorporated by reference herein.

### TECHNICAL FIELD

The disclosure relates to a filtration system. More specifically, the invention relates to an alternating tangential flow filtration system having an improved level sensor assembly for filtering fluids, particularly biological fluids comprising cells.

### BACKGROUND

Accurate level detection and control are important for several applications, particularly, for example, for certain types of filtration systems. Filtration is typically performed to separate, clarify, modify and/or concentrate a fluid solution, mixture or suspension. In the biotechnology and pharmaceutical industries, filtration is vital for the successful production, processing, and testing of new drugs, diagnostics and other biological products. For example, in the process of manufacturing biologicals using cell culture, particularly animal cell culture, filtration is done for clarification, selective removal and concentration of certain constituents from the culture media or to modify the media prior to further processing. Filtration can also be used to enhance productivity by maintaining a culture in perfusion at high cell concentration.

The application describes an improved fluid filtration system having an improved level sensor, which can be used to significantly enhance the performance, robustness and consistency of the filtration.

### BRIEF SUMMARY

In one general aspect, the application describes a filtration system comprising:
(1) an expansion chamber, comprising a first end and an opposing second end and a length extending between the first and second ends; and
(2) a first sensor assembly and a second sensor assembly mounted on the outer surface of the expansion chamber to monitor a level of fluid within the expansion chamber, wherein:
   (i) the first sensor assembly is located proximate the first end of the expansion chamber;
   (ii) the second sensor assembly is located proximate the second end of the expansion chamber;
   (iii) each of the first and second sensor assemblies includes an emitting part and a receiving part, the receiving part detects an empty chamber signal when there is no fluid between the respective receiving part and emitting part in the expansion chamber, and the receiving part detects a filled chamber signal when there is fluid between the respective receiving part and emitting part in the expansion chamber; a trigger point between the empty chamber signal and the filled chamber signal is set to control the flow direction of a fluid within the expansion chamber such that the fluid fluctuates between an upper limit and a lower limit of the expansion chamber;
      wherein:
(A)the trigger point is set to be significantly different, preferably 25-35% lower or higher, than the empty chamber signal;
(B) the first sensor assembly is longitudinally offset from the upper limit by a distance which is 15% to 25% of the length of the expansion chamber, and the direction of the offset being away from the first end of the expansion chamber; and/or
(C) the flow direction of a fluid within the expansion chamber is changed after a time delay after the first or second sensor assembly detects a signal that crosses the trigger point.

The application also describes applications and methods of using the filtration system.

Other aspects, features and advantages of the invention will be apparent from the following disclosure, including the detailed description of the invention and its preferred embodiments and the appended claims.

### BRIEF DESCRIPTION OF THE DRAWINGS

FIG. 1 shows a piping and instrumentation diagram (P&ID) of the alternating tangential flow filtration system provided with a sensor assembly and gas flow controller system;

FIG. 2 shows an expansion chamber provided with a sensor assembly according to an embodiment of the present invention;

FIG. 3A shows an alternating tangential flow filtration system according to an embodiment of the application;

FIG. 3B shows another view of the expansion chamber of the alternating tangential flow filtration system shown in FIG. 3A;

FIG. 4A provides a graphical representation of the operation of an upper level sensor assembly according to an embodiment of the present invention;

FIG. 4B provides a graphical representation of the operation of a lower level sensor assembly according to an embodiment of the present invention;

FIG. 5A provides a graphical representation of sensor values of a conventional alternating tangential flow filtration system, the unit of the sensor value (Y-axis) is negative centibel (-cB);

FIG. 5B provides a graphical representation of sensor values of an alternating tangential flow filtration system according to an embodiment of the present invention, the unit of the sensor value (Y-axis) is -cB;

FIG. 6A is a photograph showing biomass buildup in an expansion chamber of a Pneumatic Alternating Cell Separator (PACS) on day 8 of a mammalian cell cultivation using a conventional sensor assembly; and

FIG. 6B is a photograph showing the disappearance of biomass buildup in an expansion chamber using an improved sensor assembly according to an embodiment of the application.

### DETAILED DESCRIPTION

The following description is presented to enable a person of ordinary skill in the art to make and use the various embodiments. Descriptions of specific compositions, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

Filtration systems for biological fluids were described previously in the art. One type of filtration system previously developed is known as an alternating tangential flow (ATF) filtration system. Pneumatic Alternating Cell Separator (PACS), a specific type of ATF filtration system, is described in U.S. Pat. No. 8,845,902, the content of which is incorporated herein by reference in its entirety.

An exemplary PACS is illustrated in FIG. 1, which is identical to FIG. 1 of U.S. Pat. No. 8,845,902. The PACS includes a process vessel (1) connected to a filtration module (6) having a filter element (8), an entrance end (7) and an exit end (9). The exit end of the filtration module is connected to an expansion chamber (17), and the expansion chamber, in turn, is connected to a gas flow controller (28). The gas flow controller alternately provides for positive and negative pressure (e.g., compressed air and vacuum) into the expansion chamber, allowing for the fluid contained in the process vessel (1) to be alternately aspirated through the filter element (8) into the expansion chamber (17) and expelled from the expansion chamber, through the filter element, and back into the vessel. By doing so, the system creates an alternating tangential flow of fluid, such as a liquid cell culture or a fluid containing the contents of lysed cells (i.e., cell lysate), through the filter element.

The expansion chamber (17) is provided with two level sensors (25 and 26) to determine the fluid level in the expansion chamber and provide feedback to the gas flow controller (28), which, in turn, actuates the alternating positive and negative pressure cycle in the expansion chamber. The level sensors can be removeably mounted to the expansion chamber. The sensor is designed to direct a signal, such as a microwave signal, originated from an emitting part of the sensor through a window in the expansion chamber. The signal can be reflected off a surface in the expansion chamber opposite the window and collected by a receiving part. The signal can also be collected by a receiving part mounted onto a second window opposite the first window through which the signal is transmitted. When liquid is present in the expansion chamber at or above the level of the emitting part and receiving part, the signal is changed. For example, in case of a microwave signal, the signal is attenuated as it passes through the liquid. The signal can also be increased when different measurement techniques are used, for example when a reflected signal is measured. Sensor electronics compare the received signal to a predetermined threshold level and provide an output indicating the presence or absence of liquid at the sensor level.

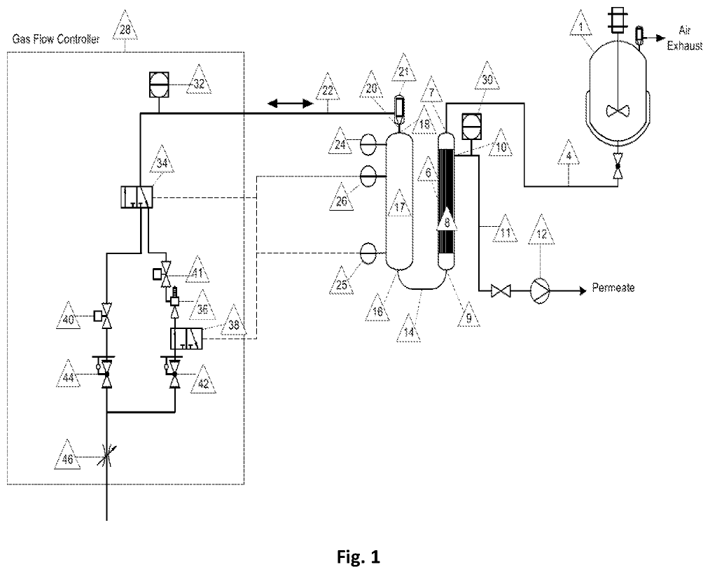

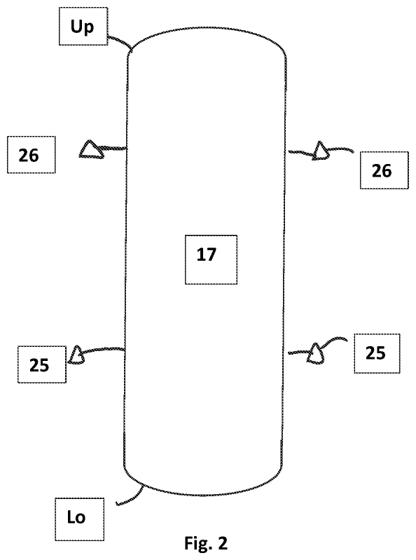

Referring to FIG. 2, the level sensor assembly is provided on expansion chamber 17. The expansion chamber 17 has a first or upper end Up and an opposing second or lower end Lo, and a length extending between the first and second ends. The expansion chamber 17 is provided with a first or upper sensor assembly 26 and a second or lower sensor assembly 25. Each of the sensor assemblies 25 and 26 independently comprises a signal emitting part and a signal receiving part and the two parts are preferably positioned on the outer surface of the expansion chamber opposite each other. The upper sensor assembly 26 is preferably provided proximate the upper end Up of the expansion chamber 17 and the lower sensor assembly 25 is preferably provided proximate the lower end Lo.

The first and second sensor assemblies are level sensors used to monitor and control the fluid level within the expansion chamber 17. Each level sensor controls the flow of fluid within the expansion chamber 17 by sending a signal, including but not limited to, a microwave signal, from its emitting part to the respective receiving part. When a portion of the expansion chamber 17 is empty and there is no fluid between the two parts of the sensor assembly 25 or 26, the receiving part will receive a first signal or predetermined signal, hereinafter referred to as an empty chamber signal. Depending on the sensor used, the empty chamber signal can be any suitable value in view of the present disclosure. For example, the empty chamber signal can be approximately 650 negative centibel (-cB) for a sensor that emits a microwave signal. However, when the expansion chamber 17 is filled up and fluid is present between the emitting and receiving parts of the sensor 25 or 26, it is expected that the receiving part will receive a second signal, hereinafter referred to as a filled chamber signal, which is dampened or reduced compared to that of the empty chamber signal. Depending on the sensor used, the value of the empty chamber signal, and the property of the fluid (e.g., the composition or density of the cells in the fluid), the filled chamber signal can be any suitable value that is different from the empty chamber signal. For example, when a microwave based measuring technique is used, the filled chamber signal can be approximately 300 -cB for a sensor that emits a microwave signal with a 650 -cB empty chamber signal. The difference in the values of the empty chamber signal and the filled chamber signal is used to control the flow of fluid in the expansion chamber 17 by use of a trigger point or threshold that is between the empty chamber signal and filled chamber signals.

When the trigger point is crossed at the upper sensor assembly 26 from empty chamber signal to filled chamber signal, fluid is drawn out of the expansion chamber 17. When the trigger point is crossed at the bottom sensor assembly 25 from filled chamber signal to empty chamber signal, fluid is drawn back into the expansion chamber 17. When the liquid is aspirated out of the storage vessel, negative pressure is applied until the liquid drawn into the expansion chamber reaches an upper limit level (UL). The upper level sensor compares the received signal with a pre-determined trigger point or threshold to detect the presence of the liquid at the UL. When the threshold value is crossed, the upper level sensor triggers a switch of the gas flow controller (28) from negative to positive pressure cycle to apply positive pressure. The positive pressure is then applied until the liquid is expelled from the expansion chamber and the liquid level in the chamber drops to a lower limit level (LL). The lower level sensor compares the received signal with a pre-determined trigger point or threshold to detect the drop of the liquid to the LL. When the threshold value is crossed, the lower level sensor triggers a switch of the gas flow controller (28) from positive to negative pressure cycle to apply negative pressure and start another cycle. Note however, when the trigger point is crossed again at the upper sensor assembly 26 from filled chamber signal to empty chamber signal while the fluid moves down, or when the trigger point is crossed again at the bottom sensor assembly 25 from empty chamber signal to filled chamber signal while the fluid moves up, nothing happens to the gas flow controller.

The trigger point is conventionally set at a value that is slightly lower than the empty chamber signal. The upper level sensor is conventionally mounted on the outer surface of the expansion chamber at about the UL and the lower level sensor is conventionally mounted on the outer surface of the expansion chamber at about the LL. The fluid contained within the expansion chamber typically fluctuates between the UL and the LL to provide a target fluid displacement volume to the filtration system. Such a system has applications in perfusion of cultured animal cells, as well as other varied filtration applications.

However, in practice, it was found that cell culture biomass tends to build up on the inside surface of the expansion chamber, particularly at or near the UL, where the upper level sensor locates. Depending on the type of sensor (and the measurement principle of the sensor) applied, the cell culture biomass buildup, in turn, can interfere with the sensor signal and impair the ability of the upper level sensor to function properly, and thus have a negative impact on the overall operation of the filtration system.

In addition, because the lower level sensor is installed above the low end of the expansion chamber, the liquid column is stopped at this level when the liquid is expelled from the expansion chamber, resulting in a holdup volume of liquid in the lower portion of the expansion chamber (17) and the U bow (14) connected to the filtration module (6). Because the liquid is a cell suspension outside of the controlled environment of the process vessel, it is preferred to minimize this holdup volume of liquid.

It was discovered in the present invention that lowering the trigger point or threshold of the upper level sensor provided more accurate detection of the UL, thus more reliable control of the gas flow controller. It was further discovered in the present invention that while biomass would accumulate and get progressively worse with time on the surface of the expansion chamber around the UL where the upper level sensor positioned, biomass was not found significantly at a new position lower than that area due to the washing effect created by greater flux of alternating liquid flow at the lower position. Additionally, a time delay can be implemented before the gas controller 28 actuates the application of positive or negative pressure to maintain the target cell culture displacement volume and/or to minimize the holdup volume of liquid in the lower portion of the expansion chamber (17) and the U bow (14) connected to the filtration module (6).

Accordingly, in one general aspect, the application relates to an improved level sensor assembly for use in a filtration system for a fluid, wherein the level sensor has a trigger point or threshold that is set at a value significantly lower than the empty chamber signal. In one embodiment, the sensor is a microwave sensor, and the trigger point is set to be 25-35%, such as 25%, 26%, 27%, 28%, 29%, 30%, 31%, 32%, 33%, 34% or 35%, lower than the empty chamber signal. Other sensors can also be used in the invention, such as sensors based on light scattering, sensors based on capacity measurements, and the like. The trigger point or threshold for such other sensors can be determined using methods known in the art in view of the present disclosure. In one embodiment, the sensor detects a reflected signal, and the trigger point is set to be 25-35%, such as 25%, 26%, 27%, 28%, 29%, 30%, 31%, 32%, 33%, 34% or 35%, higher than the empty chamber signal.

In one embodiment of the application, the sensor is a microwave sensor, and there is a difference from 160 -cB to 200 -cB between the trigger point for the sensor assemblies 25, and 26 and the empty chamber value, and more preferably a difference of approximately 175 cB. For example, where the empty chamber value is 650 -cB, the trigger point is preferably about 450-cB to 490-cB, such as 450-cB, 455cB, 460-cB, 465-cB, 470-cB, 475-cB, 480-cB, 485-cB, or 490-cB, and more preferably approximately 475-cB.

In one embodiment, a filtration system of the application uses a sensory means for more accurate detection of the UL, thus more reliable control of the gas flow controller.

In another general aspect, the application relates to an improved expansion chamber for a liquid filtration system, having an upper level sensor located on the outer surface of the expansion chamber at a position significantly lower than the upper limit level (UL). Preferably, the expansion chamber 17 has a maximum trigger point or threshold value. In one embodiment, the first (upper) sensor assembly 26, and more particularly the transmitting and receiving parts of the first sensor assembly 26, are longitudinally offset from UL by a distance, i.e., the upper sensor is located at a position significantly lower than the UL along the cylinder axis of the expansion chamber. In a preferred embodiment, the first sensor assembly 26, and more particularly the transmitting and receiving parts of the first sensor assembly 26, are longitudinally offset from the UL by a distance which is about 15% to 25%, such as 15%, 16%, 17%, 18%, 19%, 20%, 21%, 22%, 23%, 24% or 25%, of the length of the expansion chamber 17. The distance of the offset is preferably of such a magnitude to ensure that the location where the biomass is building up (e.g., the UL) is sufficiently spaced apart from the location of the upper sensor assembly 26, such that there is no or minimal interference in the sensor signal caused by the accumulated biomass. The exact distance of the offset is scale-dependent. In one embodiment, the distance of the offset is about 3.5 to 5.5 inches, such as about 3.5, 4, 4.5, 5 or 5.5 inches. In one embodiment, the distance of the offset is more preferably approximately 4.5 inches. The direction of the offset is away from the upper end of the expansion chamber 17, such that in terms of the longitudinal axis of the expansion chamber, the first sensor assembly 26 is positioned lower than the UL by a distance which is between 15% and 25% of the length of the expansion chamber 17 (e.g., approximately 4.5 inches).

In one embodiment, a filtration system of the application uses a means to sufficiently space apart the location of the biomass build up from the location of the upper sensor assembly 26, such that there is no or minimal interference in the sensor signal caused by the accumulated biomass.

In another general aspect, the application relates to an improved liquid filtration system comprising a time delay mechanism after either the first or second sensor assembly 26 or 25 is triggered (i.e., once the signal sensed by either sensor assembly 26 or 25 crosses the trigger point), before the fluid is either drawn out of or into the expansion chamber 17. In one embodiment, the time delay is implemented after a sensor is triggered by a signal crossing a trigger point or threshold that is set at a value significantly lower than the empty chamber signal, to thereby maintain a target cell culture displacement volume. In another embodiment, the time delay is implemented after an upper level sensor located on the outer surface of the expansion chamber at a position significantly lower than the upper limit level (UL), to thereby maintain a target cell culture displacement volume. In a further embodiment, the time delay is implemented after the lower sensor is triggered to thereby reduce the volume between the lower sensor and the lower end of the hollow fibers, to thus minimize the number of cells that remain outside the process vessel in uncontrolled conditions at the end of the pressure cycle. Depending on the scale of the system, such as the overall cycle time, and other factors such as the type of application, the desired crossflow rates, etc., the time delay can vary. In certain embodiments, the time delay is about 1000 ms to 1300 ms, such as 1000 ms, 1100 ms, 1200 ms, 1300 ms, or any value in between, and preferably approximately 1200 ms.

In one embodiment, a filtration system of the application uses a means to reduce the volume between the lower sensor and the lower end of the hollow fibers, to thus minimize the number of cells that remain outside the process vessel in uncontrolled conditions at the end of the pressure cycle.

The invention will now be described in further detail in the context of an exemplary filtration system, and more particularly a pneumatic alternating cell separator system that has applications in perfusion of cultured animal cells. However, it will be understood that the inventive sensor assembly is not limited to such applications, and in particular could be used in a similar manner as described below on any alternating tangential flow filtration system.

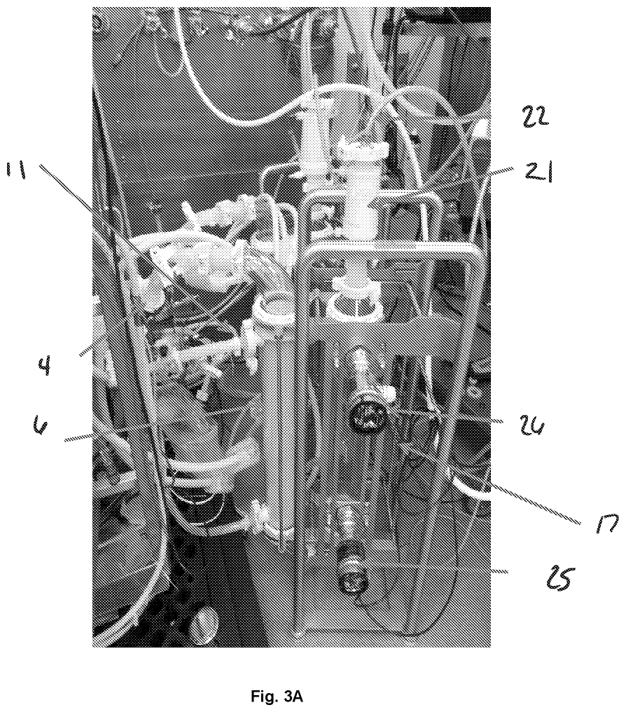

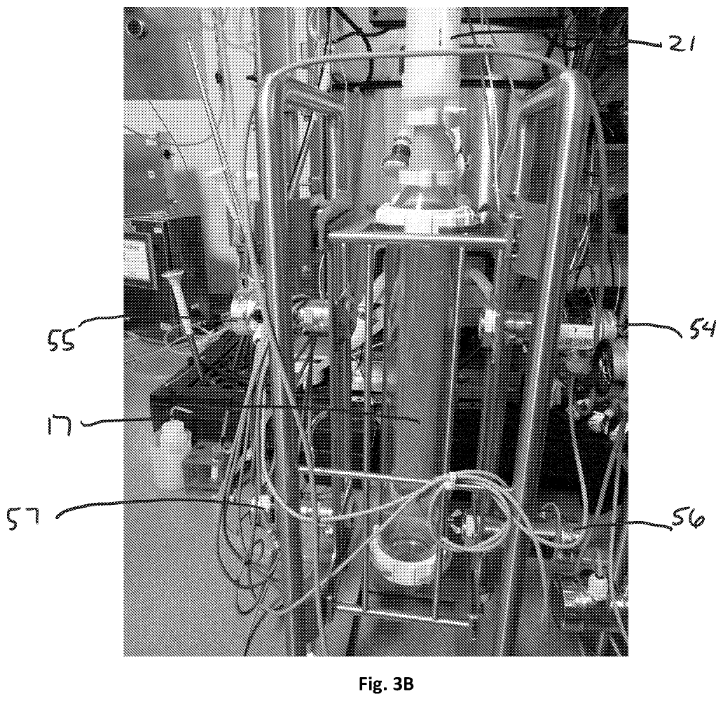

Referring to FIGS. 3A and 3B there is shown a filtration system comprising a process vessel 1, an expansion chamber 17, a filtration module 6, and at least one gas flow controller 28.

The process vessel 1 can be any suitable container for a fluid to be filtered. It will be understood that the term “fluid” is used interchangeably herein with the term “liquid” for describing the fluid transferred between the process vessel 1 and the expansion chamber 17. The process vessel 1 can be, for example, a bioreactor, a fermenter or any other vessel, nonexclusively including vats, barrels, tanks, bottles, flasks, containers, and the like that can contain liquids. The process vessel 1 may be composed of any suitable material such as Ultra Low Density Poly Ethylene (ULDPE), Low Density Polyethylene (LDPE), multilayer materials like the CX5-14 film, polyester, tie barrier layer, Ethyl Vinyl Alcohol (EVOH), and a Polyester Elastomer (PE), or a multilayer material that contains PET, PA, EVOH and ULDPE, metal such as stainless steel, glass, or the like.

In one embodiment, the process vessel 1 is connected to the filtration module 6 by a fluid transfer line or conduit 4, such that fluid is directed from the process vessel 1 into the filtration module 6 via an entrance end 7 of the filtration module 6. In one embodiment, one end of the fluid transfer line 4 is connected to the process vessel 1, optionally by a valve (not shown), and the other end is connected to a port formed at the entrance end 7 of the filtration module 6, optionally by a valve (not shown). In one embodiment, the fluid transfer line 4 comprises a tubing. Preferably, the tubing is kept as short as possible to minimize the holdup volume of the liquid.

Suitable ports nonexclusively include any sanitary, leak-proof fittings known in the art such as a compression, standard Ingold or a sanitary-type fitting. Suitable joints nonexclusively include pipes, tubes, hoses, hollow joint assemblies, and the like. The joint can vary from one system to another, based on the configuration and requirements of the vessel and process. In a preferred embodiment, the fluid transfer line 4 is connected to the entrance end 7 of the filtration module 6 via a tube connection, such as silicone rubber, C-flex, bioprene or dry-to-dry aseptic connections. The fluid transfer line 4 can also be connected to the process vessel 1 and the filtration module 6 by means of valves and suitable clamps, such as a triclamp sanitary fitting or the like. This does not preclude the use of other appropriate connections.

In addition to the entrance end 7, the filtration module 6 has an exit end or retentate end 9 and a permeate port or fluid harvest port 10 which allows for recovery or harvesting of the permeate. In certain embodiments, the fluid filtration system further comprises at least one permeate pump 12 or filtrate pump connected to the permeate port 10. The retentate exit end 9 of the filtration module 6 is connected to the expansion chamber 17, for example, by a fluid transfer line 14 or a dry-to-dry aseptic connection. Preferably, the fluid transfer line 14 is in the form of a tube assembly but other types of connectors are suited as well.

Suitable materials for the filtration module 6 include, but are not limited to, plastics like polysulfone, metal or glass. In preferred embodiments, materials appropriate for gamma sterilization and preferably commonly used as disposable materials (i.e., generally for one-time use) are suitable materials. One skilled in the art knows what materials are commonly used and suitable for this application. Most preferably, the filtration module 6 is made out of disposable material, and preferred examples include, but are not limited to, polysulfone, polyethersulfone and modified polyethersulfone. The filtration module 6 comprises a filter 8. Suitable filter elements include, but are not limited to, hollow fiber filters, mesh filters, screen filters, and the like.

Most preferably, the filter element 8 is a hollow fiber filter or filters consisting of a screen mesh. Suitable hollow fiber filtration membranes or screen filters are commonly available from various vendors, e.g., ready-to-process hollow fibers from GE Healthcare or WaterSep, Krosflo hollow fibers from Spectrum, and Microza hollow fibers from Pall. In certain preferred embodiments, the filter 8 is positioned and extends longitudinally from the entrance end 7 to the exit end 9 of the filtration module 6, which enables tangential flow of the fluid along the filter 8. When the filter 8 is a hollow fiber filter, the axes of the hollow fibers preferably extend longitudinally from the entrance end 7 to the exit end 9 of the filtration module 6.

Also, where filter 8 is a hollow fiber filter, both ends, the entrance end and the exit end of filter 8 are sealed against the housing wall of the filtration module 6 to prevent mixing of the retentate side, and the permeate (filtered) side of the filter 8. The retentate side of the fiber 8 is the lumen side of the hollow fiber and the permeate (or filtrate) side is the shell side of the hollow fiber. Such a leak proof seal can be formed by a number of methods known in the art, including O-rings, gaskets or any other means that form an impenetrable barrier between the circumference at each end of the filter 8 and the inner wall of the housing.

The expansion chamber 17 can be any type of container having any type of shape such as, e.g., a cylindrical, square, or circular shape (not limiting). In certain embodiments, the expansion chamber 17 has a cylindrical shape. However, the expansion chamber 17 must be suited for containing both the fluid provided from the process vessel 1 and the gas provided from the gas flow controller 28 (for example, through a gas line 22).

The expansion chamber 17 is preferably made, at least in part (e.g., comprising a “window”) or substantially completely, from a transparent material in order to visualize the liquid level in the chamber 17. Suitable materials for the expansion chamber 17 include, but are not limited to, plastics such as polysulfone, polyethersulfone and modified polyethersulfone. Alternatively, the expansion chamber 17 is made of metal such as stainless steel. In preferred embodiments, materials that are appropriate for gamma sterilization are used as suitable materials. One skilled in the art would know what materials are commonly used and suitable for this application.

The expansion chamber 17 has a first end 16 and an opposing second end 18, and a length extending between the first and second ends 16, 18. The expansion chamber 17 is connected to the exit end 9 of the filtration module 6 on one side and to a gas flow controller 28 on the other side. More particularly, the first end 16 (also referred to herein as the entrance end) of the expansion chamber 17 includes a first opening through which fluid flows from the exit end 9 of the filtration module 6. The second end 18 (also referred to herein as the exit end) of the expansion chamber 17 includes a second opening and is operably connected to the gas flow controller 28 by a gas line 22.

In a preferred embodiment, the gas line 22 is a reversible inlet/exhaust line. In other embodiments, separate inlet and exhaust gas lines are provided (not shown). Preferably, the gas line 22 comprises a sterile filter 21 in order to provide for sterile gas, e.g., compressed air, into the expansion chamber 17, thereby minimizing the risk of contaminating the liquid phase in the expansion chamber 17. In a preferred embodiment, the sterile filter 21 is an air filter, which preferably is provided with a heater in order to prevent blockage of the filter due to wetting by vapor generated in the expansion chamber 17. When the gas line 22 comprises a sterile filter 21, the filter is further connected to the expansion chamber 17 by an additional gas line 20.

In operation of the filtration system, the fluid is alternatively and repeatedly aspirated and received from the process vessel 1 through the filter 8 into the expansion chamber 17 and expelled from the expansion chamber 17 back into the process vessel 1 through the filter 8. More particularly, the gas flow controller 28 alternately provides for positive and negative pressure through the gas line 22 into the expansion chamber 17, allowing for the fluid contained in the process vessel 1 to be alternately aspirated through the filter element 8 into the expansion chamber 17 and expelled from the expansion chamber 17, through the filter 8, and back into the vessel 1.

The positive pressure, which is defined as higher pressure than the pressure in the filtration module 6, is preferably obtained by feeding a gas, such as compressed air (from a supply source), through the gas line 22. Instead of compressed air, other gases or gas mixtures may be used, e.g., nitrogen, nitrogen/oxygen or nitrogen/oxygen/carbon dioxide mixtures and the like. The negative pressure, which is defined as a lower pressure than the pressure in the filtration module 6, is generated in the controller, for instance, by creating a vacuum. The negative pressure is preferably obtained by applying under-pressure or vacuum into the expansion chamber 17. The vacuum can be generated by any known system or method for creating under-pressure in the expansion chamber 17, such as a vacuum pump, a vacuum injector and the like. In a preferred embodiment, however, the gas flow controller 28 does not require a separate vacuum supply.

In this way, an alternating tangential flow of fluid is generated through the filter 8 between the process vessel 1 and the expansion chamber 17. The tangential flow can be harvested through the fluid harvest port 10 into a permeate line 11. In a preferred embodiment, the permeate line 11 comprises a permeate pump 12 which regulates the permeate flow, controls the removal of filtered fluid permeate from the system, and serves as a check valve to regulate the unrestricted flow of permeate from the filtration module 6. The tangential flow (more commonly known as crossflow) is regulated by the PACS controller, i.e. gas flow controller 28. Pressure in the permeate line can be monitored by a pressure sensor 30, as shown in FIG. 1. Alternating flow of retentate between the expansion chamber 17 and process vessel 1 is through the lumen side of the filter 8 in the filtration module 6. In operation, the expansion chamber 17 comprises a direct gas-liquid interface, without separation means, that is formed by the liquid contained in the system, which is in direct contact with the gas phase provided by the gas flow controller 28.

In certain embodiments, the gas flow controller 28 can comprise a pressure-measuring device 32, such as a pressure sensor, which serves to monitor and/or regulate the pressure in the gas line 22. In addition, the gas flow controller 28 can comprise a pressure-measuring device 30, which serves to measure the pressure in the permeate line 11. In certain embodiments, the gas flow controller 28 is connected to an air or other gas supply, which provides the gas flow controller with air or gas, from which the pressure can optionally be reduced with a pressure reducer 46. The gas that may be reduced in pressure is further directed either through a pressure controller 44 and control valve 40 toward the gas line 22 in order to provide for the positive pressure or, alternatively, through a pressure controller 42, vacuum injector 36 and control valve 41 in order to provide for negative pressure into the gas line 22 and the expansion chamber 17.

The first level sensor assembly 26 and the second level sensor assembly 25 monitor the liquid level in the expansion chamber 17 and provide feedback to the gas flow controller 28. The gas flow controller 28, in turn, actuates the alternating positive and negative pressure cycle in the expansion chamber 17. Referring to FIG. 3B, the first level sensor assembly 26 includes a signal generator or emitting part 54 and a detector or receiving part 55, and the second level sensor assembly 25 includes a signal generator or emitting part 56 and a detector or receiving part 57. The respective emitting parts 54, 56 are positioned opposite of the respective receiving parts 55, 57. Referring to FIGS. 3A-3B, the first sensor assembly 26 is preferably provided proximate the upper end 18 of the expansion chamber 17, and the second sensor assembly 25 is preferably provided proximate the lower end 16. More particularly, the emitting part 54 and the receiving part 55] of the first sensor assembly 26 are preferably located proximate the (upper) second end 18 of the expansion chamber 17 and opposite of each other, such that the first level sensor assembly 26 monitors the UL of the liquid level in the expansion chamber 17. The emitting part 56 and the receiving part 57 of the second level sensor assembly 25 are preferably located proximate the (lower) first end 16 of the expansion chamber 17 and opposite of each other, such that the second level sensor assembly 25 monitors the LL of the liquid level in the expansion chamber 17. The emitting parts 54, 56 and receiving parts 55, 57 are preferably mounted on an exterior surface of the expansion chamber 17.

Level sensors as such are known in the art and can use a variety of parameters to measure the level of liquid in the expansion chamber 17, e.g., sensors based on light scattering, sensor based on capacity measurements, microwave sensors and the like. In some embodiments, the sensor assemblies 26, 25 are microwave sensors, such that the emitting part 54 or 56 of each assembly 26 or 25 sends a microwave signal to the respective receiving part when the liquid level reaches the UL or LL and crosses a trigger point as described in greater detail below.

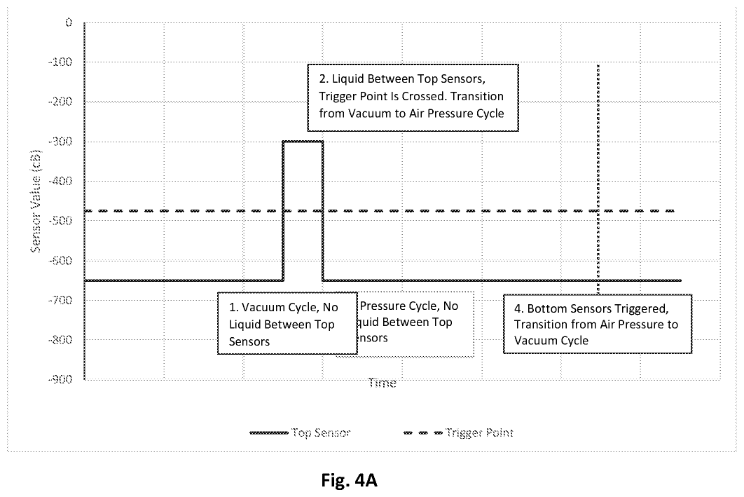

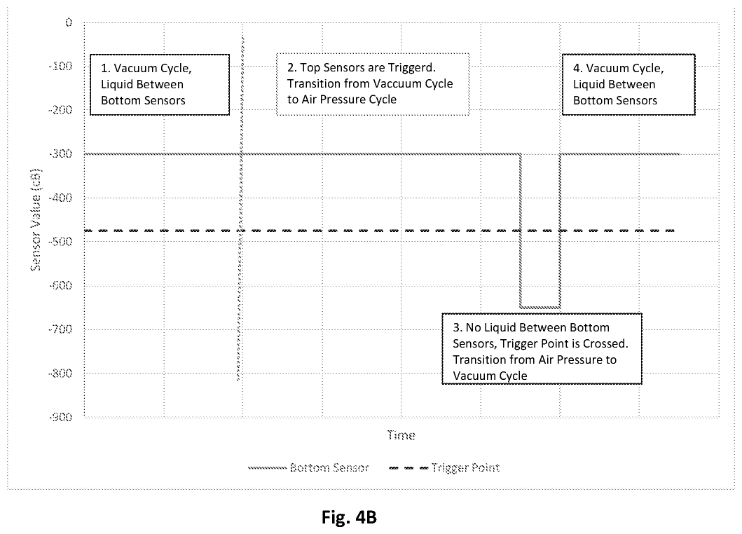

Referring to FIGS. 4A-4B, with an exemplary PACS, when the expansion chamber 17 is empty, and more particularly when there is no fluid, namely cell culture, between the sensors of each sensor assembly 26 or 25, it is expected that the receiving part will read a first signal or empty chamber signal for example, approximately 650 -cB. However, when the expansion chamber 17 fills up and cell culture is present between the sensor assemblies 26, 25, the signal is attenuated, and the expected value is changed by more than 50% in log scale relative to the empty chamber signal (e.g., approximately 300-cB), hereinafter referred to as a filled chamber signal. The difference in the receiving part values is used to control the flow of fluid in the expansion chamber 17 by use of a threshold or trigger point signal which is between the empty chamber and filled chamber signals. In a preferred embodiment, there is a difference of 160-cB to 200-cB, and more particularly a difference of approximately 175-cB, between the trigger point signal for the sensor assemblies 26, 25 and the empty chamber signal for a microwave sensor. For example, where the empty chamber signal is 650-cB, the trigger point signal is preferably between 450-cB and 490-cB, and more preferably approximately 474-cB.

Preferably, the first (upper) sensor assembly 26, and more particularly the emitting part 54 and receiving part 55 of the first sensor assembly 26, are longitudinally offset from the UL by a distance. In an exemplary PACS design, the first sensor assembly 26, and more particularly the transmitting and receiving parts 54, 55 of the first sensor assembly 26, are longitudinally offset from the UL by a distance which is about 15% to 25% of the length of the expansion chamber 17. The offset is negative in a direction away from the upper end, such that the UL is above the upper level sensor. The predetermined distance is preferably of such a magnitude to ensure that the location where the biomass is building up (i.e., the UL) is sufficiently spaced apart from the location of the emitting part 54 and receiving part 55 of the first sensor assembly 26, such that there is no or minimal interference in the sensor signal caused by the accumulated biomass. In one embodiment, the distance of the offset is preferably between 3.5 and 5.5 inches. In one embodiment, the distance of the offset is more preferably approximately 4.5 inches. The direction of the offset is away from the upper end 18 of the expansion chamber 17, such that in terms of the longitudinal axis of the expansion chamber 17, the first sensor assembly 26 is positioned lower than the UL by a distance of between 15% and 25% of the length of the expansion chamber 17 (e.g., approximately 4.5 inches).

In a preferred embodiment, once either the first or second sensor assembly 26, 25 is triggered (i.e., once either sensor assembly 26, 25 crosses the trigger point signal), there is preferably a time delay before the gas controller 28 actuates the application of positive or negative pressure to maintain the target fluid displacement volume. The desired duration of the time delay is dependent on scale, application, and desired crossflow rate. In an exemplary PACS design, the time delay is about 1000 to 1300 ms, such as about 1000, 1100, 1200 or 1300 ms, and more preferably approximately 1200 ms.

More particularly, during the filtration process, the liquid contained in the process vessel 1 is aspirated out of the vessel 1, through the filter 8, and ultimately into the expansion chamber 17, and is alternately expelled from the expansion chamber 17 back through the filter 8 and into the vessel 1. Referring to FIG. 4A, when negative pressure is applied by the gas flow controller 28 to the expansion chamber 17, the liquid is aspirated out of the process vessel 1 and drawn into the expansion chamber 17 until the first level sensor assembly 26 responds, that is, until the first sensor assembly 26 detects that the cell culture has reached the level of the upper sensor and the trigger point signal of 475 -cB is crossed (i.e. the detected signal increases from below to above the configured trigger point signal). Once the first level sensor assembly 26 detects that the trigger point signal has been achieved or surpassed and the cell culture level in the expansion chamber 17 has reached the UL, and after a time delay of approximately 1200 ms, the gas flow controller 28 is triggered to switch to apply positive pressure to the expansion chamber 17. The application of positive pressure to the expansion chamber 17 causes liquid to be expelled from the expansion chamber 17 and back into the process vessel 1, until second sensor assembly 25 detects that the cell culture level in the expansion chamber 17 has reached the LL, i.e. the signal of the second sensor assembly 25 falls below the trigger point of 475-cB. Subsequently, the gas flow controller 28 is again triggered to switch to the application of negative pressure to the expansion chamber 17 and aspiration of the liquid from the process vessel 1 into the expansion chamber 17. The switch to the application of negative pressure can occur with or without a time delay (e.g., after a time delay of approximately 1200 ms). As a result, the cell culture flows back and forth through the filter 8 in a controlled way (crossflow), allowing for permeate extraction into the permeate line 11.

In one embodiment, the gas flow controller 28 comprises a shut-off valve 38, which is functionally in contact with the first and second level sensor assemblies 26, 25 and which closes when the liquid in the expansion chamber 17 has reached the UL. The gas flow controller 28 preferably further comprises a switch-over valve 34, which is in contact with the first and second level sensor assemblies 26, 25 and which determines whether compressed air (having higher pressure than the pressure in the filter-containing compartment) or vacuum or under-pressure (as compared to the pressure in the filter-containing compartment) is applied into the gas line 22.

By adjusting the trigger point signal for the sensor assemblies 26, 25 to significantly lower values than the empty chamber signal, the system according to the present invention provides an improved buffer between the empty and full chamber sensor values. For example, in a conventional system, the trigger point for a microwave sensor can have a factory setting of 515-cB. According to an embodiment of the application, the trigger point for the microwave sensor can be set at 475-cB instead. The optimal setting of the trigger point can be dependent on the diameter of the expansion chamber, i.e. the extent of attenuation of the microwave signal by the thickness of the liquid column between emitter and receiver. In some other embodiments, for smaller PACS systems, a substantial part of the microwave signal travels around the expansion chamber. Therefore, the change in cB signal between full and empty chamber is smaller, and the optimal trigger point is thus also different.

Also, the implementation of a time delay for actuation of the positive pressure once the upper sensor assembly 26 is triggered leads to a setup where the sensor assembly is provided at an offset location relative to the UL. By this, the position of sensor assembly 26 is exposed to a high flux of cell culture flow that limits biomass buildup and hence signal interference to a minimum. Likewise, the implementation of a time delay for actuation of the negative pressure once the lower sensor assembly 25 is triggered leads to a setup where the expansion chamber 17 is emptied more completely than only to the level of sensor assembly 25. This minimizes the holdup volume of the assembly and hence minimizes exposure of cells to the conditions within the filtration system, which are less controlled than the conditions in the process vessel.

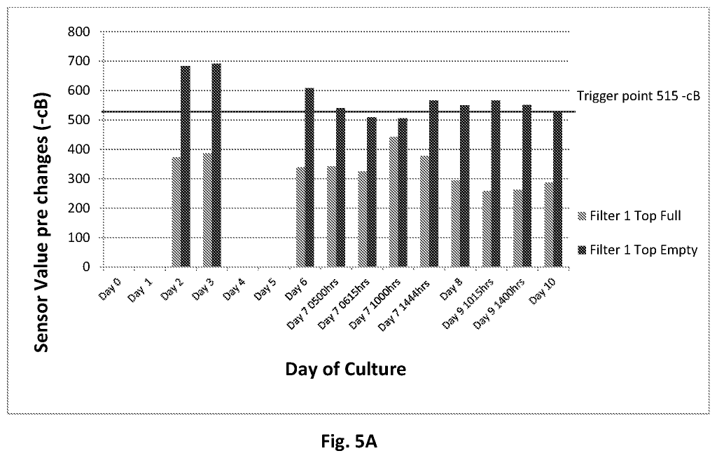

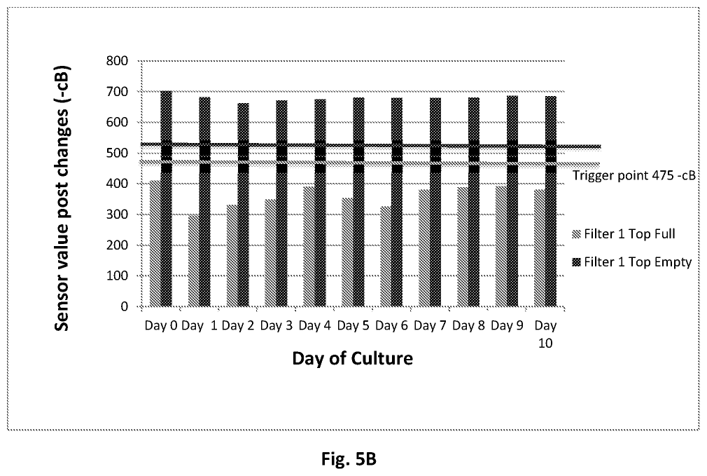

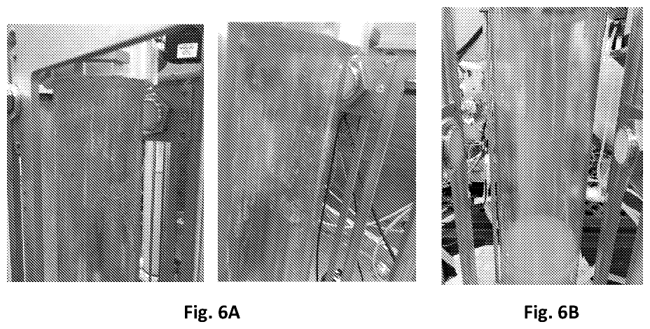

These effects are demonstrated in FIGS. 5A-5B. Referring to FIGS. 5A-5B, the “Filter 1 Top Full” represents a state of the expansion chamber 17 where the culture is present between the emitting part 54 and receiving part 55 of the first sensor assembly 26, and “Filter 1 Top Empty” represents a state of the expansion chamber 17 where the fluid level of the cell culture has lowered such that the cell culture is not present between the emitting part 54 and receiving part 55 of the first sensor assembly 26. FIG. 5A shows the “Filter 1 Top Full” and “Filter 1 Top Empty” states in a conventional system utilizing a trigger point of 515-cB, while FIG. 5B shows the “Filter 1 Top Full” and “Filter 1 Top Empty” states in an improvement to the conventional system utilizing a trigger point of 475-cB and a time delay according to an embodiment of the application. As is observed by comparing FIGS. 5A and 5B, the inventive system provides for the generation of steadier and more uniform signals by the first sensor assembly 26 (e.g., due to the application of the time delay) and provides an improved buffer between the empty and full chamber sensor values (i.e., between the “Filter 1 Top Full” and “Filter 1 Top Empty” values), even over a relatively prolonged duration. See, also FIG. 6A and FIG. 6B, which show that the area of the first sensor assembly 26 is relatively free of biomass buildup when the first sensor assembly is located at a position lower than the UL.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A filtration system comprising:
(1) an expansion chamber, comprising a first end and an opposing second end and a length extending between the first and second ends; and
(2) a first sensor assembly and a second sensor assembly mounted on the outer surface of the expansion chamber to monitor a level of fluid within the expansion chamber, wherein:
(i) the first sensor assembly is located proximate the first end of the expansion chamber;
(ii) the second sensor assembly is located proximate the second end of the expansion chamber;
(iii) each of the first and second sensor assemblies includes an emitting part and a receiving part, the receiving part detects an empty chamber signal when there is no fluid between the respective receiving part and emitting part in the expansion chamber, and the receiving part detects a filled chamber signal when there is fluid between the respective receiving part and emitting part in the expansion chamber; a trigger point between the empty chamber signal and the filled chamber signal is set to control the flow direction of a fluid within the expansion chamber such that the fluid fluctuates between an upper limit and a lower limit of the expansion chamber; wherein:
(A) the trigger point is set to be 25-35% different from the empty chamber signal;
(B) the first sensor assembly is longitudinally offset from the upper limit by a distance which is 15% to 25% of the length of the expansion chamber, and the direction of the offset being away from the first end of the expansion chamber; and/or
(C) the flow direction of a fluid within the expansion chamber is changed after a time delay after the first or second sensor assembly detects a signal that crosses the trigger point.

2. The filtration system according to claim 1, wherein the first sensor assembly is longitudinally offset from the upper limit by the distance that is 15% to 25% of the length of the expansion chamber direction away from the upper end, and the flow direction of the fluid within the expansion chamber is changed after the time delay.

3. The filtration system according to claim 1, wherein the trigger point is 25-30% different from the empty chamber signal, and the flow direction of the fluid within the expansion chamber is changed after the time delay.

4. The filtration system according to claim 1, wherein the trigger point is 25-30% different from the empty chamber signal, the first sensor assembly is longitudinally offset from the upper limit by the distance that is 15% to 25% of the length of the expansion chamber direction away from the upper end, and the flow direction of the fluid within the expansion chamber is changed after the time delay.

5. The filtration system according to claim 1, wherein the time delay is 1000 ms to 1300 ms, after the first or second sensor assembly detects a signal that crosses the trigger point.

6. The filtration system according to claim 1, wherein the first sensor assembly is longitudinally offset from the upper limit by a distance of 3.5 to 5.5 inches.

7. The filtration system according to claim 1, wherein each of the upper level sensor assembly and lower level sensor assembly is independently selected from the group consisting of sensors based on light scattering, sensors based on capacity measurements, and microwave sensors.

8. The filtration system according to claim 7, wherein each of the upper level sensor assembly and lower level sensor assembly is a microwave level sensor.

9. The filtration system according to claim 8, wherein the trigger point is about 150-cB to 200-cB, lower than the empty chamber signal.

10. The filtration system according to claim 9, wherein the empty chamber signal is 650-cB and the trigger point is 475-cB.

11. The fluid filtration system according to claim 1, further comprising:
a process vessel containing a fluid to be filtered;
a filtration module containing a filter and having an entrance end and an exit end, the process vessel being in fluid communication with the filtration module; and
a gas flow controller; wherein
the expansion chamber is in fluid communication with the filtration module and operably connected to the gas flow controller, the gas flow controller alternatively provides the expansion chamber with positive gas pressure and negative pressure; and
when the first sensor assembly first detects a signal that crosses the trigger point, the gas flow controller is triggered to apply positive gas pressure to the expansion chamber such that fluid is drawn out of the expansion chamber and into the process vessel, after a first time delay, and
when the second sensor assembly first detects a signal that crosses the trigger point, the gas flow controller is triggered to apply negative pressure to the expansion chamber such that fluid is drawn from the process vessel into the expansion chamber, after a second time delay.

12. The fluid filtration system according to claim 11, wherein the first time delay and the second time delay are identical.

13. The fluid filtration system according to claim 11, wherein the first time delay and the second time delay are different.

14. The fluid filtration system according to claim 11, wherein each of the first time delay and the second time delay is independently 1000 ms to 1300 ms.

15. The fluid filtration system according to claim 1, wherein the negative pressure is obtained by creating a vacuum in the expansion chamber, and the positive pressure is obtained by injecting gas into the expansion chamber.

16. The fluid filtration system according to claim 11, wherein the filtration module contains a hollow fiber filter.

17. The fluid filtration system according to claim 11, wherein the filtration module and/or the expansion chamber are disposable.

18. A method for filtering a liquid, comprising filtering the liquid using fluid filtration system according to any one of claims 1 17claim 1.

19. ATho method of claim 18, comprising a) obtaining the fluid filtration system according to any one of claims 11 17claim 11, b) drawing liquid out of the process vessel through the filtration module into the expansion chamber by applying negative pressure into the expansion chamber; c) expelling the liquid from the expansion chamber through the filter back into the process vessel by applying a positive pressure into the expansion chamber; and d) removing the filtered liquid from the filtration system.

20. The method of claim 18 or 19, wherein the liquid is a liquid cell culture or a cell lysate.
